(19) United States
(12) Patent Application Publication
(10) Pub. No.: US 2025/0302077 A1
(43) Pub. Date: Oct. 2, 2025

(54) FOOD INGREDIENT CONVERSION METHOD AND FOOD INGREDIENT CONVERSION DEVICE

(71) Applicant: Panasonic Intellectual Property Management Co., Ltd., Osaka (JP)

(72) Inventors: FUMIYA WAYAMA, Osaka (JP); NORIYUKI HATSUGAI, Kyoto (JP); KIYOTAKA TSUJI, Osaka (JP); YASUAKI OKUMURA, Kyoto (JP); KUNIHIRO UKAI, Nara (JP)

(21) Appl. No.: 19/232,912

(22) Filed: Jun. 10, 2025

Related U.S. Application Data
(63) Continuation of application No. PCT/JP2023/044811, filed on Dec. 14, 2023.

(30) Foreign Application Priority Data
Dec. 27, 2022 (JP) 2022-210440

(51) Int. Cl. A23L 5/20

(52) U.S. Cl. CPC A23L 5/25; A23L 5/27

(57) ABSTRACT

A reaction vessel includes a first vessel in which a working electrode having at least one of an enzyme or a coenzyme immobilized thereon is disposed, a second vessel in which a counter electrode is disposed, and a membrane that separates the first vessel and the second vessel from each other, prevents passage of an organic compound, and has ion conductivity. A food ingredient conversion method includes activating at least one of the enzyme or the coenzyme by applying a voltage between the working electrode and the counter electrode with an external power supply, transferring a proton between the organic compound and an external liquid by an enzymatic reaction using at least one of the activated enzyme or coenzyme, and performing ion conduction by transferring the proton in the external liquid through the membrane between the first vessel and the second vessel and preventing the organic compound from transferring.

### BACKGROUND

### 1. Technical Field

The present disclosure relates to a food ingredient conversion method and a food ingredient conversion device.

### 2. Description of the Related Art

In manufacturing of food, converting organic substances which are food ingredients is widely performed for the purpose of, for example, modifying the food ingredients.

An example of a food ingredient conversion technique is a technique of using a nickel catalyst in order to hydrogenate fat and oil components serving as ingredients in manufacturing of margarine. In addition, using an enzyme in food manufacturing can also be one of such food ingredient conversion techniques.

It is known that, as a method for converting an organic compound using an enzyme, glucose can be oxidized (converted) with glucose dehydrogenase and nicotinamide adenine dinucleotide. For example, Japanese Unexamined Patent Application Publication No. 4-370755 discloses a glucose biosensor including a working electrode formed such that glucose dehydrogenase and nicotinamide adenine dinucleotide are adsorbed and immobilized on a particular electrode surface, although this is not a food ingredient conversion technique.

Japanese Unexamined Patent Application Publication No. 2007-163268 discloses an enzyme electrode including a conductive substrate and an enzyme, although this is not a food ingredient conversion technique. The enzyme is formed of an associated protein of a first enzyme such as glucose dehydrogenase and a second enzyme such as diaphorase. When glucose is allowed to act on this enzyme electrode, the glucose is oxidized (converted) by a catalytic action of glucose dehydrogenase in the presence of nicotinamide adenine dinucleotide (NAD⁺).

### SUMMARY

The configurations of the methods for converting an organic compound (hereinafter, also referred to as an organic substance) using an enzyme or devices therefor in the related art are not intended for food ingredient conversion; therefore, there is room for improvement in addressing the methods and configurations suitable for conversion of food ingredients.

As an example thereof, there is a known biosensor that involves the conversion of an organic compound using an enzyme to measure a required numerical value, such as quantification of a specific component in a system. In the case where the purpose is food ingredient conversion, it is necessary to convert a target organic compound in a large amount at low cost compared with such a biosensor, and there is room for improvement in performing more efficient conversion of the organic compound.

One non-limiting and exemplary embodiment provides a food ingredient conversion method that can achieve more efficient conversion of an organic compound. Another non-limiting and exemplary embodiment provides a food ingredient conversion device that can achieve more efficient conversion of an organic compound.

In one general aspect, the techniques disclosed here feature a food ingredient conversion method including converting an organic compound in a reaction system including an external liquid and contained in a reaction vessel, wherein the reaction vessel includes a first vessel in which a working electrode having at least one of an enzyme or a coenzyme immobilized thereon is disposed, a second vessel in which a counter electrode is disposed, and a membrane that separates an inside of the first vessel and an inside of the second vessel from each other, prevents passage of the organic compound, and has ion conductivity, and the food ingredient conversion method includes activating at least one of the enzyme or the coenzyme by applying a voltage between the working electrode and the counter electrode with an external power supply located outside the reaction vessel and connected to the working electrode and the counter electrode, transferring a proton between the organic compound and the external liquid by an enzymatic reaction using at least one of the activated enzyme or coenzyme, and performing ion conduction by transferring the proton in the external liquid through the membrane between the first vessel and the second vessel and preventing the organic compound from transferring between the first vessel and the second vessel.

According to the food ingredient conversion method and food ingredient conversion device according to aspects of the present disclosure, more efficient conversion of an organic compound can be performed.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1A is a view illustrating an example of the structure of a food ingredient conversion device according to an embodiment;

FIG. 1B is a view illustrating an example of the structure of a food ingredient conversion device according to a comparative example;

FIG. 2 is a sectional view taken along line II-II in FIG. 1A or FIG. 1B;

FIG. 3 is a schematic diagram for illustrating food ingredient conversion according to the embodiment;

FIG. 4 is a schematic diagram for illustrating food ingredient conversion according to Example 1 of the embodiment;

FIG. 5 is a flowchart illustrating a food ingredient conversion method according to Example 1 of the embodiment;

FIG. 6 is a schematic diagram for illustrating food ingredient conversion according to Example 2 of the embodiment; and

FIG. 7 is a flowchart illustrating a food ingredient conversion method according to Example 2 of the embodiment.

### DETAILED DESCRIPTIONS

### Underlying Knowledge Forming Basis of the Present Disclosure

Glucose dehydrogenase (GDH) functions as a catalyst in a D-glucose degradation reaction represented by a formula (1) below in which nicotinamide adenine dinucleotide (NADH) or nicotinamide adenine dinucleotide phosphate (NADPH) is involved as a coenzyme.

D-glucose+NAD⁺→D-glucono-1,5-lactone+NADH+H⁺ Formula (1)

The reaction represented by the formula (1) is a reaction in which a proton (H⁺) is released (transferred) from D-glucose, which is an organic compound serving as a food ingredient, by an enzymatic reaction in the presence of an enzyme to convert the D-glucose into D-glucono-1,5-lactone. However, in the formula (1), the proton balance does not match between the right side and the left side strictly because protons are supplied from water molecules, etc. in the system. In the case of using NADPH instead of NADH, NAD⁺ in the formula is replaced by NADP⁺.

On the other hand, a peroxidase functions as a catalyst in a degradation reaction of an organic compound represented by a formula (2) below by an enzymatic reaction.

ROOR′+2e⁻+2H⁺→ROH+R′OH Formula (2)

The reaction represented by the formula (2) is a reaction in which electrons (e⁻) are donated from an electron donor to an organic compound (represented by ROOR′ as a general formula), and protons are transferred from the outside of the organic compound (for example, water molecules in the system) to the organic compound to convert the organic compound to ROH and R′OH.

The knowledge based on the formula (1) will be described below.

When the concentration of glucose in a predetermined sample is measured using the reaction represented by the formula (1), for example, the concentration of NADH generated per unit time is measured. In this case, the amount of NAD⁺ consumed for measuring the concentration of glucose is not so large. On the other hand, when glucose in a liquid containing glucose is degraded by using the above-described reaction, it may be difficult to add NAD⁺ at a concentration commensurate with the amount of glucose contained in the liquid. This is because the concentration of NAD⁺ dissolved in the liquid has an upper limit (saturated concentration). In particular, in the case of using an enzymatic reaction, since solution conditions such as the optimum temperature and the optimum pH of the enzyme are present, it is often practically impossible to sufficiently increase the concentration of NAD⁺ in accordance with the amount of glucose.

Therefore, in the above reaction, it is possible to consider a method for degrading glucose contained in a high concentration in a liquid even with the addition of a small amount of NAD⁺ by oxidizing generated NADH (that is inactive in terms of enzymatic reaction) to generate activated NAD⁺ again.

Meanwhile, according to studies conducted by the present inventors, it has been found that when glucose is sequentially degraded while NADH generated in the above-described reaction is continuously oxidized by an electrochemical method and the concentration of NAD⁺ in a liquid is maintained so as not to decrease, glucose can be degraded with high efficiency and continuously while the cost in terms of energy related to participation of NADH is kept low. The same applies to an example of the formula (2) in that a small amount of inactivated enzyme is activated by electrochemical assistance and continuously utilized. Specifically, the enzymatic reactions are continued while the reactions represented by the formulas (1) and (2) are assisted by an electrochemical method, and thus, protons can be added or released (transferred in either case) to an organic compound present in an amount relatively larger than an enzyme and/or a coenzyme even with a small amount of enzyme and/or coenzyme. In addition, these reactions can be performed at high energy efficiency. Furthermore, as a result of extensive studies, the present inventors have newly found a configuration of a more advantageous (that is, efficient) food ingredient conversion device, etc. in which, while an enzyme and/or a coenzyme is continuously activated by an electrochemical method in the above-described reactions, protons are transferred to an organic compound by an enzymatic reaction using the activated enzyme and/or coenzyme.

### Aspects of Present Disclosure

The summary of aspects of the present disclosure is as follows.

According to a first aspect of the present disclosure, there is provided a food ingredient conversion method including converting an organic compound in a reaction system including an external liquid and contained in a reaction vessel, wherein the reaction vessel includes a first vessel in which a working electrode having at least one of an enzyme or a coenzyme immobilized thereon is disposed, a second vessel in which a counter electrode is disposed, and a membrane that separates an inside of the first vessel and an inside of the second vessel from each other, prevents passage of the organic compound, and has ion conductivity, and the food ingredient conversion method includes activating at least one of the enzyme or the coenzyme by applying a voltage between the working electrode and the counter electrode with an external power supply located outside the reaction vessel and connected to the working electrode and the counter electrode, transferring a proton between the organic compound and the external liquid by an enzymatic reaction using at least one of the activated enzyme or coenzyme, and performing ion conduction by transferring the proton in the external liquid through the membrane between the first vessel and the second vessel and preventing the organic compound from transferring between the first vessel and the second vessel.

According to this food ingredient conversion method, the enzymatic reaction can be caused on the first vessel side with respect to the membrane having ion conductivity, a proton can be transferred between the organic compound and the external liquid, while the proton can transfer between the first vessel and the second vessel. Since the organic compound is less likely to transfer to the second vessel side, it is easy to cause the organic compound to remain in the first vessel. If a reaction in which a proton is transferred from the organic compound to the external liquid is caused in the first vessel and protons are electrostatically collected in the second vessel, it is possible to suppress a reverse reaction caused by a reaction between the organic compound after reaction and a proton in the first vessel, that is, a reduction in the reaction efficiency. In addition, since the organic compound is less likely to transfer to the second vessel side, if protons are electrostatically collected in the second vessel, it is also possible to suppress a reduction in the reaction efficiency due to a reaction between the organic compound and a proton in the second vessel. Furthermore, if a reaction in which a proton is transferred from the external liquid to the organic compound is caused in the first vessel and protons are electrostatically collected in the first vessel, the organic compound and a proton are considered to easily react with each other in the first vessel in view of the collision theory, etc., and thus the reaction efficiency can be increased.

According to a second aspect of the present disclosure, there is provided the food ingredient conversion method of the first aspect, wherein, in the transferring, the proton is transferred from the organic compound to the external liquid.

According to this food ingredient conversion method, if protons are electrostatically collected in the second vessel, it is possible to suppress a reverse reaction caused by a reaction between the organic compound after reaction and a proton in the first vessel, that is, a reduction in the reaction efficiency.

According to a third aspect of the present disclosure, there is provided the food ingredient conversion method of the first aspect, wherein, in the transferring, the proton is transferred from the external liquid to the organic compound.

According to this food ingredient conversion method, if protons are electrostatically collected in the first vessel, the organic compound and a proton are considered to easily react with each other in the first vessel in view of the collision theory, etc., and thus the reaction efficiency can be increased.

According to a fourth aspect of the present disclosure, there is provided the food ingredient conversion method of any one of the first to third aspects, wherein a redox coenzyme serving as the coenzyme is immobilized on the working electrode, and in the activating, an electron is transferred from the working electrode to the redox coenzyme, and the enzyme is activated by the redox coenzyme to which the electron has been transferred.

According to this food ingredient conversion method, an action such as electron transfer easily occurs between the redox coenzyme and the working electrode, and thus the reaction efficiency can be increased.

According to a fifth aspect of the present disclosure, there is provided the food ingredient conversion method of any one of the first to fourth aspects, wherein the coenzyme is a redox coenzyme, an electron mediator that transfers an electron to and from the redox coenzyme is further immobilized on the working electrode, and in the activating, an electron is transferred from the working electrode to the electron mediator, and at least one of the enzyme or the redox coenzyme is activated by the electron mediator to which the electron has been transferred.

According to this food ingredient conversion method, an action such as electron transfer easily occurs between the electron mediator and the working electrode, and thus the reaction efficiency can be increased.

According to a sixth aspect of the present disclosure, there is provided the food ingredient conversion method of the fourth or fifth aspect, wherein the redox coenzyme is NADH or NADPH.

According to this food ingredient conversion method, NADH or NADPH can be used as the redox coenzyme.

According to a seventh aspect of the present disclosure, there is provided the food ingredient conversion method of any one of the first to sixth aspects, wherein the organic compound includes at least one of a monosaccharide, a disaccharide, or a polysaccharide.

According to this food ingredient conversion method, an organic compound including at least one of a monosaccharide, a disaccharide, or a polysaccharide can be efficiently converted.

According to an eighth aspect of the present disclosure, there is provided the food ingredient conversion method of any one of the first to sixth aspects, wherein the organic compound includes an alcohol.

According to this food ingredient conversion method, an organic compound including an alcohol can be efficiently converted.

According to a ninth aspect of the present disclosure, there is provided the food ingredient conversion method of any one of the first to sixth aspects, wherein the organic compound has a disulfide bond.

According to this food ingredient conversion method, an organic compound having a disulfide bond can be efficiently converted.

According to a tenth aspect of the present disclosure, there is provided a food ingredient conversion device that converts an organic compound in a reaction system including an external liquid, the food ingredient conversion device including a working electrode on which at least one of an enzyme or a coenzyme is immobilized, a counter electrode, a reaction vessel that contains the reaction system, and a voltage applicator that applies a voltage between the working electrode and the counter electrode, wherein the reaction vessel includes a first vessel in which the working electrode is disposed, a second vessel in which the counter electrode is disposed, and a membrane that separates an inside of the first vessel and an inside of the second vessel from each other, prevents passage of the organic compound, and has ion conductivity.

According to this food ingredient conversion device, advantageous effects similar to those of the food ingredient conversion method can be produced.

According to an eleventh aspect of the present disclosure, there is provided the food ingredient conversion device of the tenth aspect, wherein the enzyme is an oxidase.

According to this food ingredient conversion device, an organic compound can be efficiently converted by an enzymatic reaction in the presence of an oxidase.

According to a twelfth aspect of the present disclosure, there is provided the food ingredient conversion device of the tenth aspect, wherein the enzyme is a reductase.

According to this food ingredient conversion device, an organic compound can be efficiently converted by an enzymatic reaction in the presence of a reductase.

According to a thirteenth aspect of the present disclosure, there is provided the food ingredient conversion device of any one of the tenth to twelfth aspects, wherein the enzyme and the coenzyme are immobilized on the working electrode.

According to this food ingredient conversion device, an action such as electron transfer easily occurs between the enzyme and coenzyme and the working electrode, and thus the reaction efficiency can be increased.

It should be noted that these general or specific aspects may be implemented as a system, a method, a device, an integrated circuit, a computer program, or a computer-readable recording medium such as a CD-ROM or may be implemented as a combination of any of a system, a method, a device, an integrated circuit, a computer program, and a recording medium.

Hereinafter, embodiments will be specifically described with reference to the drawings.

It should be noted that all of the embodiments described below are comprehensive or specific examples. Numerical values, shapes, materials, constituent elements, the arrangement positions and connection forms of constituent elements, steps, the order of steps, etc. described in the following embodiments are illustrative and are not intended to limit the scope of the claims. Of the constituent elements described in the following embodiments, those which are not recited in independent claims representing the broadest concepts are described as optional constituent elements. Furthermore, each drawing is not necessarily a precise drawing. In the drawings, configurations that are substantially identical are assigned the same reference sings, and a duplicated description thereof may be omitted or simplified.

In the following description of the drawings, the X-axis direction, the Y-axis direction, and the Z-axis direction that are orthogonal to each other will be used as appropriate. In particular, in the Z-axis direction, the positive side may be referred to as an upper side, and the negative side may be referred to as a lower side in the following description.

In the present disclosure, terms that indicate relationships between elements, such as parallel and perpendicular, terms that indicate the forms of elements, such as rectangular, and numerical values not only have their precise meanings but also have meanings that include substantially the same ranges, for example, differences of about several percent.

In the drawings of the present disclosure, broken lines indicate objects that are not visible from the surface, and boundaries between regions.

### EMBODIMENTS

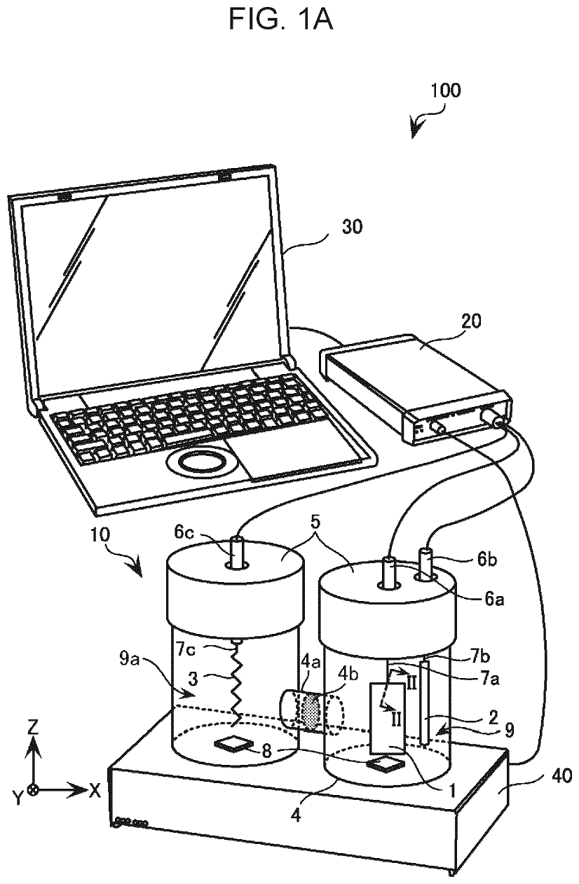

Embodiments will be specifically described below with reference to FIGS. 1A to 7.

### Food Ingredient Conversion Device

### 1. Overview

The overview of a food ingredient conversion device (hereinafter, may be simply referred to as a device) according to an embodiment will be first described with reference to FIGS. 1A and 1B. FIG. 1A is a view illustrating an example of the structure of a food ingredient conversion device according to the present embodiment. FIG. 1B is a view illustrating an example of the structure of a food ingredient conversion device according to a comparative example.

A food ingredient conversion device 100 performs electron transport between an electrode and an enzyme and/or a coenzyme upon application of a voltage to the electrode. For example, the food ingredient conversion device 100 donates electrons from an electrode to an enzyme immobilized on the surface of the electrode. This causes electron transfer between the electrode and a target molecule in a sample, and the target molecule is oxidized or reduced. For example, in the food ingredient conversion device 100, a voltage is applied to the electrode in a state where a sample (which is one example of the reaction system and is also referred to as, for example, a sample solution) containing an external liquid and target molecules is in a non-flowing state, electron transfer is thereby performed between an enzyme and/or a coenzyme immobilized on the electrode and the target molecules in the sample, and the target molecules are reduced. Subsequently, the liquid is changed from the non-flowing state to a flowing state, thereby diffusing the reduced target molecules into the liquid. By repeating the change in the flowing state of the liquid in this manner, the target molecules can be efficiently reduced throughout the liquid.

The external liquid is a main component forming the reaction system and is composed of water as a main component and various additives. The external liquid may have, in addition to a function of stabilizing, for example, the orientation of hydrophilic groups in the reaction system with a hydration bond or the like, a buffer function of suppressing an increase (or decrease) in pH when protons or the like are generated and the pH increases (or protons are consumed and the pH decreases) in the reaction system. Therefore, a buffer component composed of an acid or a base and a salt may be dissolved in the external liquid.

In this case, during the reaction, the transfer of protons occurs between target molecules and the external liquid. In the case where protons are released from target molecules, protons preferably leave the vicinity of the generated substance so as to prevent the released protons from being donated again to the substance generated after the reaction and causing the reverse reaction. On the other hand, in the case where protons are donated to target molecules, preferably, protons are rapidly supplied to the vicinity of the target substance. For this purpose, it is effective to perform stirring as described above. Meanwhile, the target substance and the substance generated after conversion also flow due to stirring. Accordingly, in the case of monotonous stirring, it may be difficult to leave protons from the vicinity of the generated substance or to supply protons to the vicinity of the target substance.

In the present disclosure, a concentration gradient of protons is formed by utilizing a property that protons tend to be attracted to the negative electrode side. In the case of releasing protons from target molecules, a region in the vicinity of the generated substance is made to be a region having a low proton concentration. On the other hand, in the case of donating protons to target molecules, a region in the vicinity of the generated substance is made to be a region having a high proton concentration. Furthermore, a reaction vessel (cell 4) is designed so that the target substance and the generated substance are likely to remain in the vicinity of the electrode on which the reaction has occurred, while such a concentration gradient is formed for protons. Specifically, a separator 4b is disposed between a working electrode (electrode 1) and a counter electrode 3 so as to separate regions in which the electrodes are disposed. The separator 4b allows protons to pass and prevents passage of the organic compounds (target substance and generated substance). Thus, protons can move between the electrode 1 and the counter electrode 3 through the separator, whereas the organic compounds remain in the region where the electrode 1 is disposed. As illustrated in FIG. 1B, in a food ingredient conversion device 100a according to a comparative example, a counter electrode 3 is disposed in the same region as that where an electrode 1 is disposed (the same region of the two regions separated from each other by a separator 4b). Unless the separator 4b is disposed between the counter electrode 3 and the electrode 1 as in the comparative example, even with a concentration gradient of protons, organic compounds can move through the entire region of the gradient. Accordingly, the generated substance moves to a region having a relatively high proton concentration, and the target substance moves to a region having a relatively low proton concentration. This is not suitable from the viewpoint of performing efficient conversion of organic compounds.

The expression “a liquid is in a non-flowing state” refers to, for example, a state in which the liquid is not stirred or shaken (that is, is not subjected to any external force such as shear force or vibration), and no movement such as shaking is observed on the liquid surface.

### 2. Structure

Next, the structure of a food ingredient conversion device 100 according to an embodiment will be described with reference to FIGS. 1A to 3.

As illustrated in FIG. 1A, a food ingredient conversion device 100 includes a stirring unit 40 that stirs a sample 9 containing target molecules (organic compound before conversion) and an external liquid to make the sample 9 in a flowing state, an electrode that performs electron transfer with the target molecules to transfer protons between the target molecules and the external liquid, an external power supply 20 that applies a voltage to the electrode, and a controller 30 that controls the external power supply 20 and the stirring unit 40.

A voltage application unit 10 is, for example, a three-electrode cell including an electrode 1 (also referred to as a working electrode), a reference electrode 2, a counter electrode 3, a cell 4, lids 5, terminals 6a, 6b, and 6c, and leads 7a, 7b, and 7c. Alternatively, the voltage application unit 10 may be, for example, a two-electrode cell including a working electrode (electrode 1) and a counter electrode 3.

The electrode 1 and the counter electrode 3 are composed of an electrically conductive substance. The electrically conductive substance may be, for example, a carbon material, a conductive polymer material, a semiconductor, or a metal.

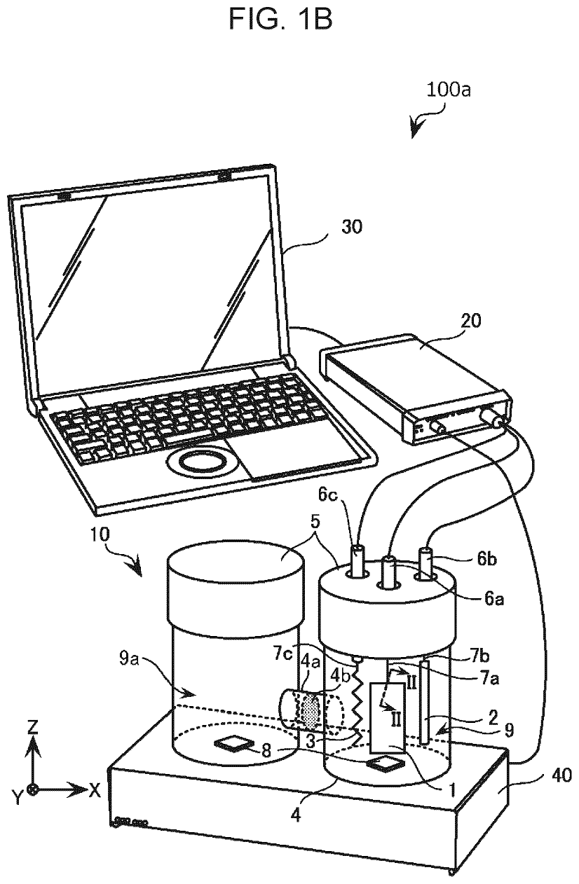

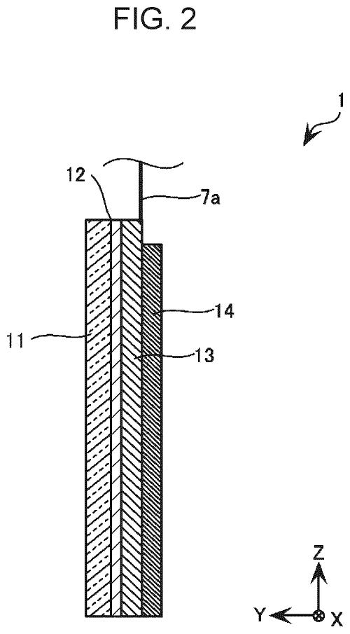

First, the electrode 1 will be described with reference to FIGS. 2 and 3. FIG. 2 is a sectional view taken along line II-II in FIG. 1A or FIG. 1B. FIG. 3 is a schematic diagram illustrating an enzyme and/or a coenzyme immobilized on an electrode, etc.

The electrode 1 is an electrode on which an enzyme and/or a coenzyme is immobilized. The electrode 1 includes, for example, a glass substrate 11, a titanium vapor-deposited layer 12 formed on the glass substrate 11 by vapor deposition, a substrate 13 formed on the titanium vapor-deposited layer 12, and a reaction layer 14 including an enzyme and/or a coenzyme immobilized on the substrate 13.

The substrate 13 may be an electrically conductive substrate composed of, for example, gold, platinum, a carbon material such as glassy carbon, graphite, or boron-doped diamond, or tin-added indium oxide (ITO: indium tin oxide). The thickness of the substrate 13 is not particularly limited.

The shape of the electrode 1 is a plate shape formed of the substrate 13, but the shape of the electrode 1 is not limited to a specific shape. The electrode 1 may be linear, plate-like, or mesh-like or may be a fiber assembly. From the viewpoint of reaction efficiency, the electrode 1 is preferably designed so as to have a large surface area and have a cross-sectional area that provides low resistance.

An electron carrier serving as a coenzyme, and an electron mediator involved in electron transfer are also immobilized on the substrate 13. However, as long as at least one of an enzyme or a coenzyme is immobilized, other substances need not necessarily be immobilized. The electron carrier is a substance that performs electron transfer between the electrode and the enzyme immobilized on the electrode and may be, for example, viologen, quinone, or indophenol.

The immobilization of the enzyme, coenzyme, and electron carrier suppresses the transfer of these molecular species from the vicinity of the electrode 1 and thus is effective in view of reaction efficiency. As a matter of course, the immobilized molecular species do not transfer to the counter electrode side (second vessel side) through the separator 4b. Thus, the immobilization of the enzyme, coenzyme, and electron carrier is effective in view of mobility of molecular species. However, for some molecular species, immobilization may result in an orientation that is not suitable for reaction. Preferably, such molecular species are not immobilized in view of reactivity. That is, the optimal configuration in this case is to immobilize molecular species with no or negligible decrease in reactivity and to immobilize molecular species with a decrease in reactivity. Even when molecular species that are not immobilized are present, the separator 4b can prevent such molecular species from transferring between a first vessel and a second vessel.

Accordingly, with respect to the freedom to choose whether immobilization is performed or not, the benefit of the separator 4b is achieved.

The enzyme and/or coenzyme immobilized on the surface of the electrode (so-called substrate 13) is a protein that oxidizes or reduces a target molecule or a coenzyme that acts cooperatively with the protein. As illustrated in FIG. 3, the enzyme, coenzyme, and electron mediator are immobilized on the electrode (substrate 13) with a chain linker. The linker includes, for example, an alkyl chain having greater than or equal to 1 and less than or equal to 14 carbon atoms. Alternatively, the enzyme, coenzyme, and electron mediator may be immobilized directly on the surface of the electrode without a linker therebetween. Note that the phrase “without a linker therebetween” means that other molecular species are not interposed between the surface of the electrode and an enzyme, a coenzyme, or an electron mediator. However, a case where a portion of the main chain or a side chain constituting an enzyme, the portion being involved in immobilization, acts like a linker is also included in the case of “without a linker therebetween”.

The reference electrode 2 is an electrode that does not react with a component in the sample 9 and maintains a constant potential and is used to control the potential difference between the electrode 1 and the reference electrode 2 to be constant by the external power supply 20. The reference electrode 2 is, for example, a silver/silver chloride electrode. Note that the food ingredient conversion device 100 may include only the electrode 1 and the counter electrode 3 without including the reference electrode 2.

The counter electrode 3 is preferably, for example, a platinum electrode in view of the ease of electron emission to maintain electrical neutrality. As the counter electrode 3, an electrically conductive substrate composed of, for example, gold, platinum, a carbon material such as glassy carbon, graphite, or boron-doped diamond, or tin-added indium oxide (ITO: indium tin oxide) may be used as in the electrode 1. The shape of the counter electrode 3 is also not limited to a specific shape. The counter electrode 3 may be linear, plate-like, or mesh-like or may be a fiber assembly. From the viewpoint of reaction efficiency, the counter electrode 3 is preferably designed so as to have a large surface area and have a cross-sectional area that provides low resistance.

In the present embodiment, the cell 4 contains the reaction system and is isolated from the outside. The cell 4 includes a first vessel (the right side of the drawing sheet) in which the electrode 1 and the reference electrode 2 are disposed, and a second vessel (the left side of the drawing sheet) in which the counter electrode 3 is disposed, the first vessel and the second vessel being connected together with a connector 4a therebetween. The separator 4b is disposed in the connector 4a so that some components of a sample 9 in the first vessel and a sample 9a in the second vessel are prevented from transferring with respect to each other. The separator used in the present embodiment is a membrane having ion conductivity.

This membrane prevents organic compounds before conversion and after conversion, an enzyme, a coenzyme, and an electron mediator from passing therethrough due to the design of the pore size. Accordingly, if, among the enzyme, coenzyme, and electron mediator, molecular species that are not immobilized are present, or if molecular species that have been immobilized but have detached are present, such molecular species do not mix with the sample 9a. In addition, since the organic compounds before conversion and after conversion, that is, the target substance and the generated substance are contained in the sample 9 to be used in the reaction, the organic compounds also do not mix with the sample 9a. Thus, the separator 4b prevents the organic compounds, the enzyme, the coenzyme, and the electron mediator from transferring from the first vessel to the second vessel.

Herein, the expression “transfer is prevented” refers to a concept including, in addition to no transfer at all, a state in which transfer is less likely to occur compared with ordinary diffusion. For example, when the amount of a component that transfers from one side to the other side through the separator 4b is less than or equal to 100 ppm, the transfer is considered to be prevented, and when the amount is less than or equal to several ppm, the transfer is considered to be prevented.

The separator 4b is not limited to a specific material as long as it has ion conductivity. The separator 4b contains, for example, a polymer having a perfluoro side chain including a sulfonic acid group. Such a structure is suitable because particular ions are likely to quickly pass through the separator 4b.

Nafion (registered trademark) is known as such a polymer. Nafion prevents the organic compounds, enzyme, coenzyme, and electron mediator from passing therethrough in terms of the pore size and has cation-selective ion conductivity. Specifically, cations such as protons are allowed to pass, while anions such as hydroxy ions, chloride ions, and phosphate ions are not allowed to pass. Accordingly, when anion species are generated on the counter electrode 3 side, unexpected problems caused by the anions passing through the separator 4b, transferring to the vicinity of the electrode 1, and reacting with the organic compounds, enzyme, coenzyme, and electron mediator are unlikely to occur. An alternative material forming the separator 4b may be porous glass or porous silicon.

The external power supply 20 applies a voltage between the electrode 1 and the counter electrode 3 of the voltage application unit 10 in accordance with a control signal output from the controller 30 and controls the potential between the electrode 1 and the reference electrode 2 to a predetermined value.

The controller 30 executes information processing for controlling the voltage application by the external power supply 20 and the movement of a motor (not illustrated) of the stirring unit 40. The controller 30 is realized by, for example, a processor, a microcomputer, or a dedicated circuit.

The stirring unit 40 controls the operation of the motor in accordance with a control signal output from the controller 30 to control the rotation speed and the rotation time of stirring elements 8 set in the cell 4.

In a method for producing an electrode on which an enzyme, a coenzyme, and an electron mediator are immobilized, first, a linker for immobilizing an organic compound, an enzyme, a coenzyme, and an electron mediator is immobilized on an electrode. The linker is dissolved in a solution of ethanol:acetonitrile=1:1, and the electrode is immersed in the resulting solution and allowed to stand for greater than or equal to one hour to immobilize the linker. The linker is preferably an alkyl chain having less than or equal to 14 carbon atoms so that electrons can transfer from the electrode to the enzyme, the coenzyme, and the electron mediator. For example, the linker is an alkyl chain having greater than or equal to 1 and less than or equal to 14 carbon atoms. In particular, the linker has, at one end thereof, a functional group capable of forming a bond with the enzyme, coenzyme, and electron mediator, such as a carboxyl group or an amino group and has, at the other end thereof, a thiol group.

Subsequently, the linker having, for example, a thiol group immobilized on the electrode through the above-described operation is bonded to the enzyme, coenzyme, and electron mediator. At this time, for example, in a case of an enzyme, an amino group at the N-terminal or in a side chain and a carboxyl group of the linker are bonded together by an amine coupling reaction. Thus, the enzyme is immobilized on the electrode with the linker therebetween. The coenzyme and the electron mediator are also immobilized on the electrode with a linker therebetween by selecting an appropriate functional group. When the enzyme, coenzyme, and electron mediator are immobilized directly on the electrode, the step of immobilizing the linker is omitted. The operations described above are merely examples. To immobilize the enzyme, coenzyme, and electron mediator on the electrode, it is possible to apply any existing technique, such as a technique of using an acid electroconductive polymer, a crosslinking agent, or the like or a technique of achieving immobilization with a monomolecular film-forming molecule therebetween.

### 3-1. Example 1

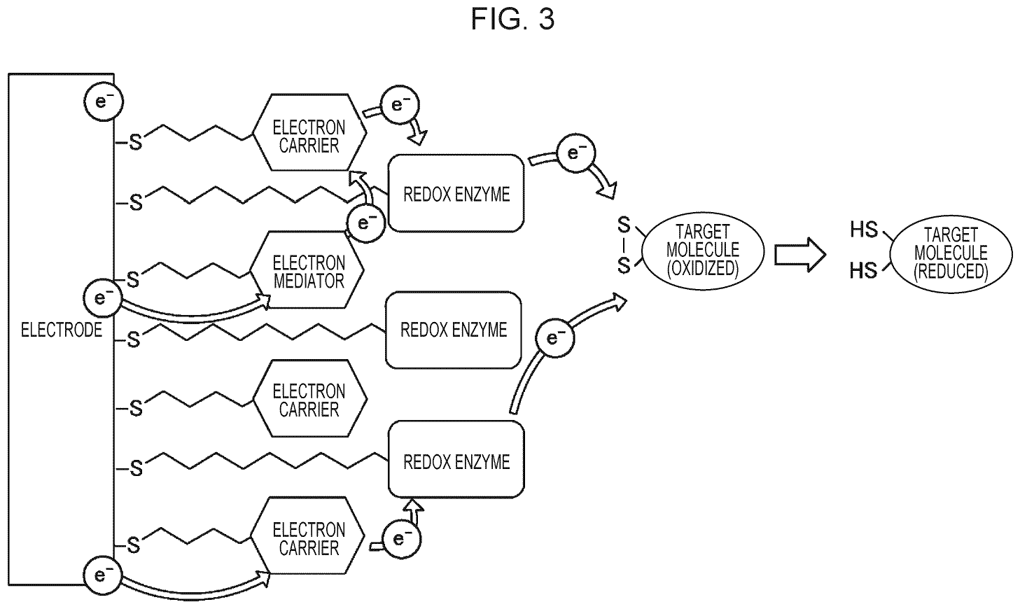

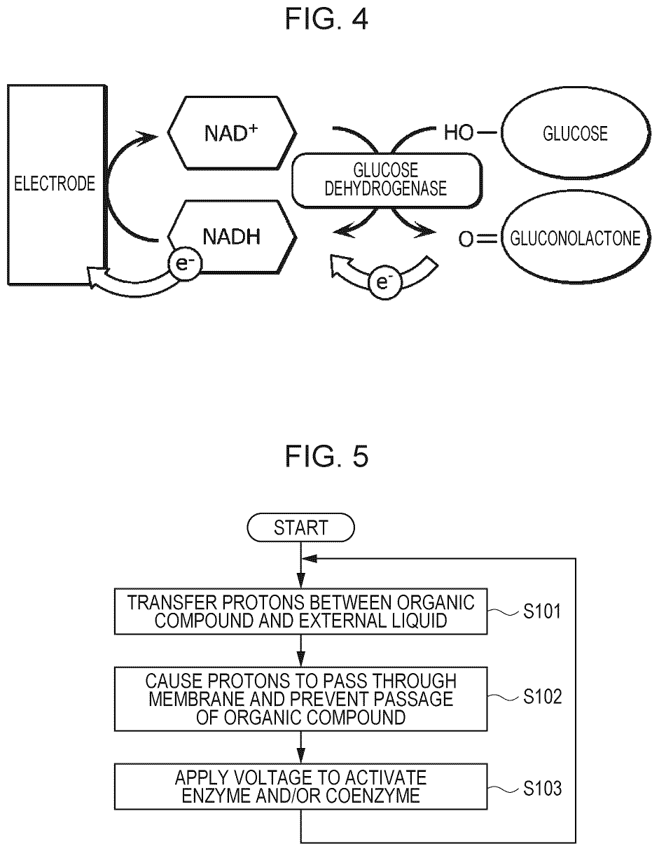

Hereinafter, an example of the degradation action of glucose by a dehydrogenase will be described as Example 1 with reference to FIGS. 4 and 5. FIG. 4 is a schematic diagram for illustrating food ingredient conversion according to Example 1 of the present embodiment. FIG. 5 is a flowchart illustrating a food ingredient conversion method according to Example 1 of the present embodiment.

In Example 1, the degradation of glucose using the reaction represented by the formula (1) is performed. Here, NAD⁺ and glucose dehydrogenase act cooperatively to cause a reaction in which a proton is released from glucose to generate gluconolactone. Accordingly, NAD⁺ is reduced and turned to NADH. In the reaction of this example, the transfer of protons between the organic compound (glucose) and the external liquid means the transfer of protons from the organic compound to the external liquid. When protons transfer from glucose to the external liquid (reaction step S101), protons accumulate in the first vessel (vessel on the electrode 1 side), and the pH increases. However, to release protons and electrons from NADH, the external power supply 20 applies a voltage between the electrode 1 and the counter electrode 3 so that the electrode 1 functions as a positive electrode and the counter electrode 3 functions as a negative electrode. Thus, the protons are attracted to the counter electrode 3 side (that is, the second vessel side) by electrostatic force and transfer through the separator 4b (membrane). At this time, gluconolactone is prevented from passing through the separator 4b and remains in the first vessel (ion conduction step S102).

Near the electrode 1, protons and electrons are released from NADH by the applied voltage to generate NAD⁺ (activation of coenzyme), and thus glucose dehydrogenase is again in the state of being capable of functioning as a catalyst (activation step S103). Repeating this cycle enables a large amount of glucose to be sequentially degraded even with a small amount of enzyme and/or coenzyme to perform an appropriate conversion of the organic compound in terms of efficiency.

### 3-2. Example 2

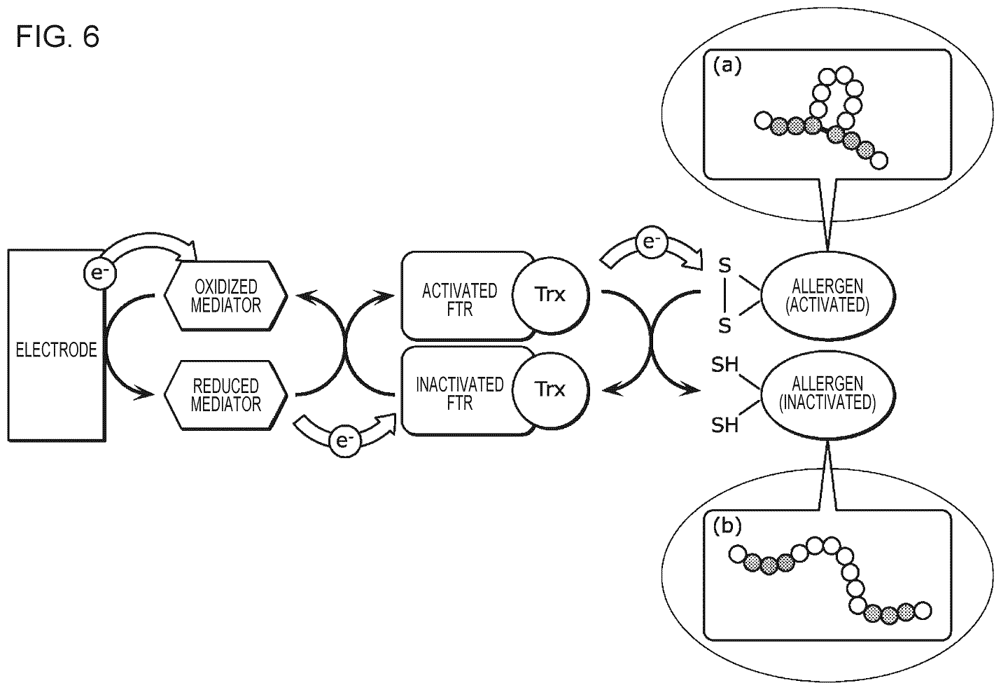

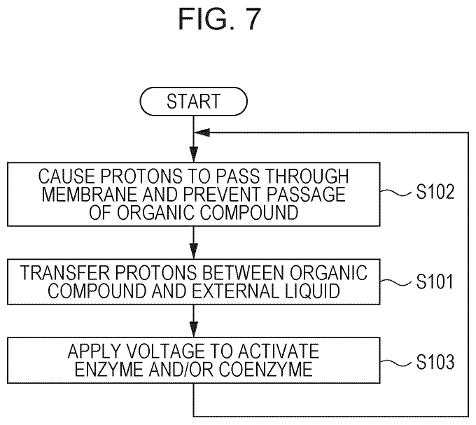

Hereinafter, an example of the reduction action of an allergen by a reductase will be described as Example 2 with reference to FIGS. 6 and 7. FIG. 6 is a schematic diagram for illustrating food ingredient conversion according to Example 2 of the present embodiment. FIG. 7 is a flowchart illustrating a food ingredient conversion method according to Example 2 of the present embodiment. In FIG. 7, steps representing the same meanings as those in the corresponding steps in FIG. 5 are assigned the same reference signs.

In Example 2, the reduction of a protein having a disulfide bond (—S—S—) is performed. Here, electrons are transferred sequentially through an electron mediator and a ferredoxin-thioredoxin reductase (FTR)-thioredoxin complex to cause a reaction in which the disulfide bond of the protein is reduced and cleaved. Accordingly, the electron mediator and the FTR-thioredoxin complex are reduced and inactivated. On the other hand, protons in the external liquid are consumed to reduce the disulfide bond to two thiol groups (—SH). In the reaction of this example, the transfer of protons between the organic compound (protein) and the external liquid means the transfer of protons from the external liquid to the organic compound. At this time, the protein is prevented from passing through the separator 4b and remains in the first vessel (ion conduction step S102). Moreover, protons collected in the first vessel are consumed to cause the above reaction in which the disulfide bond is reduced and cleaved, and the protons transfer from the external liquid to the protein (reaction step S101).

Near the electrode 1, electrons are donated to the inactivated electron mediator by the applied voltage to activate the electron mediator, and the activated electron mediator donates electrons to the inactivated FTR-thioredoxin complex to activate the FTR-thioredoxin complex, so that the FTR-thioredoxin complex is again in the state of being capable of reducing the protein (activation step S103). Repeating this cycle enables a large amount of protein to be sequentially reduced even with a small amount of enzyme and/or coenzyme to perform an appropriate conversion of the organic compound in terms of efficiency.

The protein in this example is assumed to be an allergen having a disulfide bond (—S—S—). Examples of allergenic proteins having disulfide bonds (hereinafter also simply referred to as allergenic proteins) include allergens derived from food such as beans, wheat, milk, seafood, eggs, and rice, environments such as pollen, animals, nematodes, filamentous fungi, molds, and ticks, and animals such as animal furs and dandruff. In particular, in the case of considering food, typical examples of the allergenic protein include prolamins, ovalbumin, and β-lactoglobulin.

The disulfide bond in the allergen is reduced to thiol groups (—SH) as electrons are donated from an activated FTR. FIG. 6 schematically illustrates, above the allergen (activated), a portion (a) containing a disulfide bond of the allergen before reduction. FIG. 6 also schematically illustrates, below the allergen (inactivated), a portion (b) in the allergen after reduction corresponding to (a) above. Open circles illustrated in (a) and (b) each indicate one amino acid residue, and hatched circles each indicate an amino acid residue that the IgE antibody recognize (epitope). As illustrated in (a) and (b), when the disulfide bond of the allergen is reduced, a loop portion of the amino acid sequence is loosened, and thus a different amino acid sequence is inserted in the middle of the amino acid sequence that the IgE antibody recognizes. This increases the possibility that the IgE antibody can no longer recognize the specific binding site. Thus, the allergenicity of the allergen is weakened.

Note that, although a loop portion is described as an example in (a) and (b) above, the portion is not limited to this. In a three-dimensional structure of a protein, a functional site is formed as amino acid residues located far from each other in the amino acid sequence of the peptide chain of the protein come close to each other. Therefore, when the disulfide bond is reduced, the linkage between secondary structures of the protein that has been linked together through the disulfide bond breaks, and the functional site formed by the linkage between the secondary structures is no longer maintained. More specifically, a disulfide bond is a bond that links secondary structures of the protein to each other to further strengthen the three-dimensional structure of the protein. Accordingly, when the disulfide bond is cleaved, the linkage between the secondary structures breaks, and the flexibility (fluctuation) of the three-dimensional structure of the allergenic protein increases. As a result, the functional site of the allergenic protein (for example, a conformational epitope) is less likely to be maintained; therefore, this increases the possibility that the IgE antibody can no longer recognize the specific binding site of the allergenic protein.

Furthermore, as a result of an increase in the flexibility (fluctuation) of the three-dimensional structure of the allergenic protein due to the reduction of the disulfide bond, digestive enzymes are likely to act on the cleaved sites of the peptide chains of the allergenic protein cleaved by the digestive enzymes; therefore, the allergenic protein is more easily digested by the digestive enzyme.

The description has been given above, and the organic compound treated by the food ingredient conversion device 100 may be an organic compound including not only glucose, which is a monosaccharide, but also disaccharides such as sucrose (cane sugar, sugar), lactose (milk sugar), and maltose (malt sugar), and polysaccharides such as starch, and is not limited to a specific raw material. For example, a trace amount of glucose contained in egg white, etc. can also be selectively degraded. Moreover, an appropriate enzyme, coenzyme, electron mediator, and the like suitable for the raw material to be converted can be selected. For example, when glucose is converted into gluconolactone, glucose dehydrogenase and NADH can be used as the enzyme and the coenzyme, respectively.

The organic compound treated by the food ingredient conversion device 100 may be an organic compound containing an alcohol, and the reaction formulas in such a case are as shown in formulas (3), (4), and (5) below. Here, a reaction (conversion) in which an alcohol dehydrogenase is used as an enzyme, and an aldehyde is generated with NAD⁺ to decrease an alcohol can be performed.

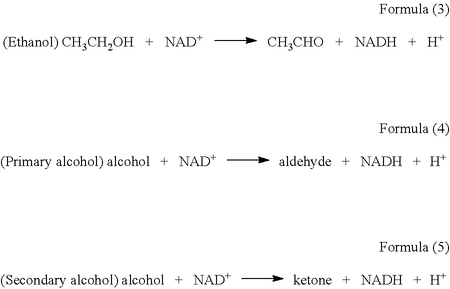

The food ingredient conversion method and the food ingredient conversion device according to the present disclosure have been described above on the basis of embodiments, but the present disclosure is not limited to these embodiments. Various modifications of the embodiments that are conceivable by those skilled in the art and other embodiments obtained by combining some of the constituent elements in the embodiments are also included in the scope of the present disclosure so long as they do not depart from the spirit of the present disclosure.

The food ingredient conversion method and the like according to the present disclosure are useful for improving the efficiency in food ingredient conversion.

What is claimed is:

1. A food ingredient conversion method comprising converting an organic compound in a reaction system including an external liquid and contained in a reaction vessel,
wherein the reaction vessel includes a first vessel in which a working electrode having at least one of an enzyme or a coenzyme immobilized thereon is disposed, a second vessel in which a counter electrode is disposed, and a membrane that separates an inside of the first vessel and an inside of the second vessel from each other, prevents passage of the organic compound, and has ion conductivity, and
the food ingredient conversion method includes
activating at least one of the enzyme or the coenzyme by applying a voltage between the working electrode and the counter electrode with an external power supply located outside the reaction vessel and connected to the working electrode and the counter electrode,
transferring a proton between the organic compound and the external liquid by an enzymatic reaction using at least one of the activated enzyme or coenzyme, and
performing ion conduction by transferring the proton in the external liquid through the membrane between the first vessel and the second vessel and preventing the organic compound from transferring between the first vessel and the second vessel.

2. The food ingredient conversion method according to claim 1,
wherein, in the transferring, the proton is transferred from the organic compound to the external liquid.

3. The food ingredient conversion method according to claim 1,
wherein, in the transferring, the proton is transferred from the external liquid to the organic compound.

4. The food ingredient conversion method according to claim 1,
wherein a redox coenzyme serving as the coenzyme is immobilized on the working electrode, and
in the activating,
an electron is transferred from the working electrode to the redox coenzyme, and
the enzyme is activated by the redox coenzyme to which the electron has been transferred.

5. The food ingredient conversion method according to claim 1,
wherein the coenzyme is a redox coenzyme,
an electron mediator that transfers an electron to and from the redox coenzyme is further immobilized on the working electrode, and
in the activating,
an electron is transferred from the working electrode to the electron mediator, and
at least one of the enzyme or the redox coenzyme is activated by the electron mediator to which the electron has been transferred.

6. The food ingredient conversion method according to claim 4,
wherein the redox coenzyme is NADH or NADPH.

7. The food ingredient conversion method according to claim 1,
wherein the organic compound includes at least one of a monosaccharide, a disaccharide, or a polysaccharide.

8. The food ingredient conversion method according to claim 1,
wherein the organic compound includes an alcohol.

9. The food ingredient conversion method according to claim 1,
wherein the organic compound has a disulfide bond.

10. A food ingredient conversion device that converts an organic compound in a reaction system including an external liquid, the food ingredient conversion device comprising:
a working electrode on which at least one of an enzyme or a coenzyme is immobilized;
a counter electrode;
a reaction vessel that contains the reaction system; and
a voltage applicator that applies a voltage between the working electrode and the counter electrode,
wherein the reaction vessel includes
a first vessel in which the working electrode is disposed, a second vessel in which the counter electrode is disposed, and a membrane that separates an inside of the first vessel and an inside of the second vessel from each other, prevents passage of the organic compound, and has ion conductivity.

11. The food ingredient conversion device according to claim 10,
wherein the enzyme is an oxidase.

12. The food ingredient conversion device according to claim 10,
wherein the enzyme is a reductase.

13. The food ingredient conversion device according to claim 10,
wherein the enzyme and the coenzyme are immobilized on the working electrode.
